(19) United States
(12) Patent Application Publication
(10) Pub. No.: US 2008/0009044 A1
(43) Pub. Date: Jan. 10, 2008

(54) Biocatalytic oxidation process useful in the manufacture of moxidectin

(75) Inventors: Daniel Howard Cohen, Princeton, NJ (US); Thomas Gerard Cullen, Milltown, NJ (US); Jignesh Patel, Plainsboro, NJ (US); Michael Joseph O'Neill, Allentown, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/825,491

(22) Filed: Jul. 5, 2007

Related U.S. Application Data
(60) Provisional application No. 60/818,815, filed on Jul. 6, 2006.

(51) Int. Cl. C12P 17/18

(52) U.S. Cl. 435/119

(57) ABSTRACT

The present invention provides a regioselective biological process for the oxidation of the 23-hydroxy group of LLF-28249-α which comprises reacting LL-F28249-α with a biocatalyst that is capable of specifically oxidising the 23-hydroxy group of LL-F28249-α.

The present invention also provides the use of the regioselective biological oxidation process in the manufacture of moxidectin.

This application claims the benefit under 35 U.S.C. §119(e) to co-pending U.S. provisional application No. 60/818,815, filed Jul. 6, 2006, which is hereby incorporated by reference in its entirety.

### BACKGROUND OF THE INVENTION

Moxidectin (23-methoxime-LL-F-28249-α) is a potent endectocidal agent. Procedures to prepare moxidectin are disclosed in, for example U.S. Pat. No. 4,988,824 and U.S. Pat. No. 6,762,327. Known methods for the manufacture of moxidectin utilize a chemical synthesis starting from the natural product LL-F28249-α, which consists of 4 steps. One step is an oxidation procedure which requires protection of the 5-hydroxy group of LL-F28249-α. Oxidizing agents which may be used in this manufacturing step, in many instances, on a manufacturing scale, require large amounts of pyridine and a corrosive catalyst, such as dichloroacetic acid, or involve oxidizing agents, which on a manufacturing scale, may introduce unwanted risks. To achieve widespread commercial availability of moxidectin, the synthetic conversion from LL-F28249-αshould be energy efficient, economical, safe and environmentally sound.

Therefore, it is an object of this invention to provide an improved oxidation process for the production of moxidectin.

It is another object of this invention to provide an oxidation process, which affords mild reaction conditions and high product yields.

It is a feature of this invention that the oxidation process utilizes fewer chemicals and demonstrates enhanced ecological safety.

It is another feature of this invention that the process may be economic and have minimal environmental impact.

These and other objects and features of the invention will become more apparent from the detailed description set forth hereinbelow.

### SUMMARY OF THE INVENTION

The present invention provides a one step process for the preparation of 23-keto-LL-F28249-α which comprises reacting LL-F28249-α with a biocatalyst that is capable of specifically oxidising the 23-hydroxy group of LL-F28249-α.

Also provided is the use of the one step oxidation process in the manufacture of moxidectin.

### DETAILED DESCRIPTION OF THE INVENTION

Moxidectin is a potent broad-spectrum endectocide of the macrocyclic lactone antimicrobial class. The unique activity of moxidectin against endo- and ectoparasites in both humans and animals, along with its high margin of safety, has had a tremendous impact on the control of internal and external parasites in companion animals and livestock. Therefore, availability of this compound is highly desired. Moxidectin is the 23-oxime derivative of LL-F28249-α. Procedures for the manufacture of moxidectin from LL-F28249-α are disclosed in, for example U.S. Pat. No. 4,988,824 and U.S. Pat. No. 6,762,327. Said procedures include an oxidation step wherein the oxidizing agents disclosed are chemical reagents, which are generally not selective and consequently require additional steps, such as protection and deprotection steps, in order to obtain the desired moxidectin product from the LL-F28249-α starting material. Further, some common difficulties encountered in using chemical reagents, such as long reaction times, difficult workup procedures, possible use of a large excess of the oxidizing agent, and the like, can be problematic on a commercial manufacturing scale.

Surprisingly, it has now been found that a regioselective biological oxidation process may be used to selectively oxidize LL-F28249-α to the corresponding 23-ketone compound under mild reaction conditions, with high product yield and without the hazardous chemical properties generally associated with conventional chemical oxidizing agents. Advantageously, said process eliminates the need for additional manufacturing steps to accommodate the protection and subsequent deprotection of the 5-hydroxy group of the LL-F28249-α starting material.

Accordingly, the present invention provides a process for the preparation of 23-keto-LL-F28249-α which comprises reacting LL-F28249-α with a biocatalyst that is capable of specifically oxidising the 23-hydroxy group of LL-F28249-α to give the 23-keto-LL-F28249-α compound. The reaction is shown in flow diagram I.

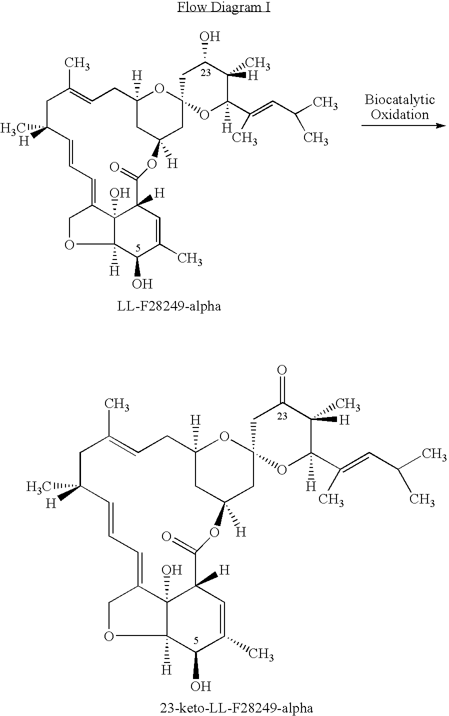

Within the scope of the present invention, the term “biocatalyst” is meant to include: a) A living microorganism (whole cell), for example in the form of vegetative cells, resting cells or freeze-dried cells;

b) The spores of said microorganism;

c) A dead microorganism, preferably in a partially disintegrated form, i.e., with the cell wall/cell membrane mechanically or chemically permeabilized;

d) A crude extract of the cell contents of said microorganism; or

e) An enzyme that converts LL-F28249-α into 23-keto-LL-F28249-α, said enzyme may be purified, partially purified, extract, cloned and expressed or wild-type.

Microorganisms suitable for use in the process of the invention include Bacillus stearothermophilus (Tetrahedron Letters, 1995, 36 (3), pp, 441-442); Rhodococcus ruber DSM 44541 (Tetrahedron: Asymmetry, 2003, 14, pp. 275-280); Arthrobacter sp., Alcaligenes bronchisepticus, Geotrichum candidum, Nocardia corralina B-276, Pseudomonas aeruginosa, Pseudomonas paucimobilis, Rhodococcus equi IFO 3730, Yarrowia lipolytica sp. (Advanced Synthesis & Catalysis, 2004, 346, pp. 125-142); or the like, preferably Rhodococcus ruber DSM 44541. Suitable whole cell microorganisms may be wild-type or clones. Whole cell microorganisms suitable for use in the inventive process may be obtained by screening biocatalytically active Streptomyces strains for the ability to oxidize LL-F28249-α to 23-keto-LL-F28249-α or by engineering whole cells to specifically oxidize LL-F28249-α to 23-keto-LL-F28249-α.

Enzymes suitable for use in the process of the invention include purified, partially purified, extract, cloned and expressed or wild-type cytochrome p450 monooxygenase enzyme (Applied and Environmental Microbiology, 2005, 71, pp. 6977-6985); alcohol dehydrogenase (Current Opinion in Chemical biology, 2004, 8, pp. 120-126); alcohol oxidases such as sec-AOx, glucose oxidase, pyranose-2-oxidase, glycolate oxidase, cholesterol oxidase, vanillyl AOx, or the like (Advanced Synthesis & Catalysis, 2004, 346, pp. 125-142); Baker's yeast (Tetrahedron Letters, 1993, 35, pp. 883-884); or the like, preferably alcohol dehydrogenase, more preferably alcohol dehydrogenase from Rhodococcus ruber DSM 44541. It is understood that when employing whole cells, and preparations thereof, in the process of the invention, the enzyme or enzyme system can be unknown.

In the process of the invention, hydrogen acceptors such as acetone and co-factors such as NADH or NADPH, may be used to facilitate oxidation, enhance catalytic effectiveness and optimize product yield. In one embodiment, the process of the invention includes the presence of hydrogen acceptors and co-factors.

In actual practice, the process of the invention is carried out by using a microorganism as the biocatalyst, which microorganism is capable of specifically oxidising the 23-hydroxy group of LL-F28249-α to give the 23-keto-LL-F28249-α. Preferably, said microorganism is cultured in a suitable cultivation medium promoting microbial proliferation and under controlled conditions in the presence of LL-F28249-α, and maintaining the joint incubation of said microorganism and its substrate for a time sufficient for the oxidation reaction to occur, until about 25% to 99.9%, preferably about 50% to 99.9%, more preferably about 80% to 99.9%, of the LL-F8249-α has been converted into the 23-keto-LL-F28249-α compound.

Alternatively, the inventive process may be carried by initially culturing a microorganism that is capable of specifically oxidising the 23-hydroxy group of LL-F28249-α to give the 23-keto-LL-F28249-α in a suitable cultivation medium promoting micorbial proliferation and under controlled conditions, and then harvesting the biomass of the microorganism by applying suitable methods such as, for example, filtration or centrifugation. The biomass of the microorganism may be either used immediately as a biocatalyst for the conversion of LL-F28249-α to 23-keto-LL-F28249-α or may be stored at reduced temperatures. Said biomass may be stored as is or after freeze-drying or spray-drying. Said microorganism, either freshly harvested or stored as described, and LL-F28249-α are then jointly incubated in a reaction medium which does not favor microbial proliferation for a time sufficient for the oxidation reaction to occur, until about 25% to 99.9%, preferably about 50% to 99.9%, more preferably about 80% to 99.9%, of the LL-F8249-α has been converted into the 23-keto-LL-F28249-α compound.

In addition to vegetative cell structures, microbial spores may be used which spores are harvested from the microorganism that is capable of specifically oxidising the 23-hydroxy group of LL-F28249-α to give the 23-keto-LL-F28249-α, and are then incubated with LL-F8249-α for a period of time that is sufficient for the oxidation reaction to take place. The incubation of spores and substrate is preferably carried out in the absence of culture medium in order to prevent the spores from germinating.

The incubation of the biocatalyst used, within the scope of the present invention, with LL-F28249-α for the specific oxidation of the alcohol at position 23 to give 23-keto-LL-F28249-α can be carried out with the aid of processes such as those customary in applied microbiology. In addition to the use of shake cultures, various fermenter systems that have long been established in microbiological research and industrial production are suitable. Types of reactors that are suitable for the process of the invention include, for example, stirred vessel reactors, loop-type reactors, bed reactors, fluidised bed reactors, membrane reactors, and special forms of a reactor, i.e. sieve-stirred reactors, rhomboid reactors, tube reactors or the like, preferably stirred vessel reactors.

The 23-keto-LL-F8249-α product may be readily separated from the reaction mixture by means of customary separation techniques, for example by extraction, filtration, fractional crystallisation or by chromatography or the like. Chromatography includes, column chromatography, thick layer chromatography or thin layer chromatography using solid support systems such as silica gel or organic exchanger resins or chiral columns. Chromatography also includes liquid-liquid chromatography, high performance liquid chromatography, or the like.

Advantageously, the process of the invention may be used in the manufacture of moxidectin. Accordingly, the present invention provides a process for the manufacture of moxidectin which comprises reacting LL-F28249-α with a biocatalyst that is capable of specifically oxidising the 23-hydroxy group of LL-F28249-α to give the 23-keto-LL-F28249-α compound; and reacting said compound with methoxylamine or a salt thereof. The process of the invention is shown in flow diagram II.

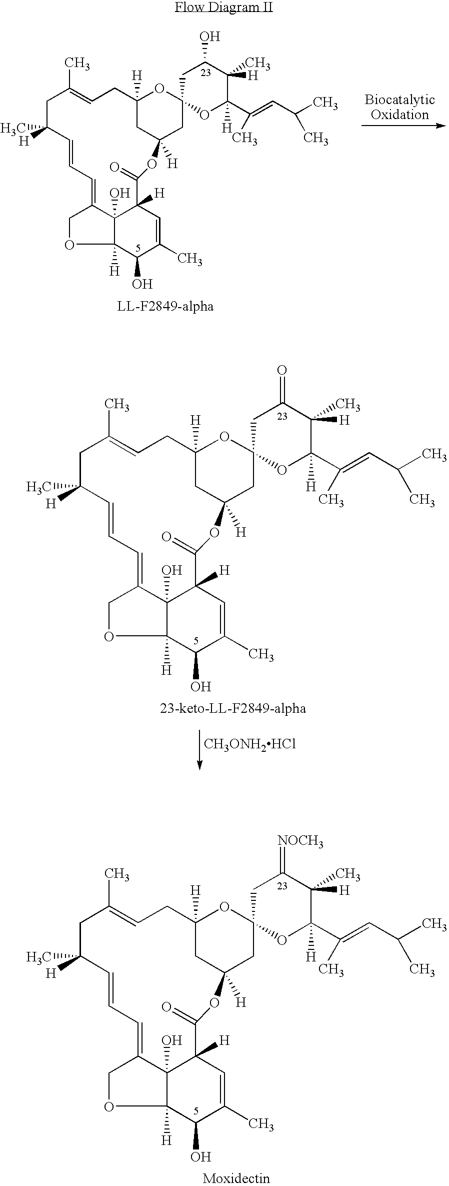

In actual practice, a microorganism that is capable of specifically oxidising the 23-hydroxy group of LL-F28249-α to give the 23-keto-LL-F28249-α compound, either freshly harvested or stored as described hereinabove, and LL-F28249-α are jointly incubated in a reaction medium which does not favor microbial proliferation for a time sufficient for the oxidation reaction to occur, until about 25% to 99.9%, preferably about 50% to 99.9%, more preferably about 80% to 99.9%, of the LL-F8249-α has been converted into the 23-keto-LL-F28249-α compound; the 23-keto-LL-F28249-αcompound (either isolated and purified or as a solution of the crude reaction product in an organic solvent such as toluene) is reacted with an aqueous solution of methyloxyamine or a salt thereof and sodium acetate to give the desired moxidectin compound; and the desired moxidectin compound is isolated from the organic phase using standard procedures such as concentration and filtration or removal of the solvent.

In order to facilitate a further understanding of the invention, the following examples are presented primarily for the purpose of illustrating more specific details thereof. The invention is not to be limited thereby except as defined in the claims.

Unless otherwise noted, all parts are parts by weight.

### EXAMPLE 1

### Preparation and Cultivation of Bacillus stearothermophilus

Production of Bacillus stearothermophilus is achieved by inoculation of the organism in a synthetic culture medium containing for 1 L of water: bactotryptone (20 g), yeast extract (10 g), saccharose (40 g), K₂SO₄ (2.6 g), and Na₂HPO4.2H₂O (6.4 g), adjusted to pH 7.1 with KOH (6N).

### EXAMPLE 2

### Preparation and Cultivation of Rhodococcus ruber DSM 44541

Rhodococcus ruber DSM 44541 (also known as Norcardia H8) are grown in shake-flask cultures at 30° C. using the following growth medium: yeast extract (10 g dm⁻³), peptone (10 g dm⁻³), glucose (10 g dm⁻³), NaCl (2 g dm⁻³), MgSO₄.7H₂O (0.147 g dm⁻³), NaH₂PO₄ (1.3 g dm⁻³), and K₂HPO₄ (4.4 g dm⁻³). Cells are harvested after ˜24-40 h by centrifugation (5000 g; 20 min; 25-30 g dm⁻³ wet cells), re-suspended in Tris-HCl buffer (0.05 M, pH 8.0), centrifuged again, and lyophilized.

### EXAMPLE 3

### Preparation of 23-(keto)-LL-F28249α (Oxidation of LL-F8249-α with Bacillus stearothermophilus in Heptane)

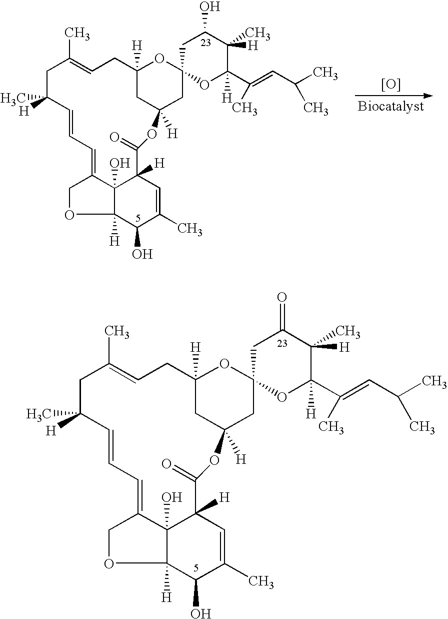

Bacillus stearothermophilus ATCC 2027 culture (30 mL), grown for 48 h at 39° C., is centrifuged (3000 g) and water is removed by decantation. Heptane (10 mL) and LL-F8249-α (40 mg) is added to the cells and the suspension is vigorously stirred at 39° C. for 24 h. After filtration and concentration under reduced pressure the crude material is used directly or purified on silica gel to yield the expected 23-keto-LL-F8249-α.

### EXAMPLE 4

### Preparation of 23-(keto)-LL-F28249-α (Oxidation of LL-F8249-α with Rhodococcus ruber DSM 44541)

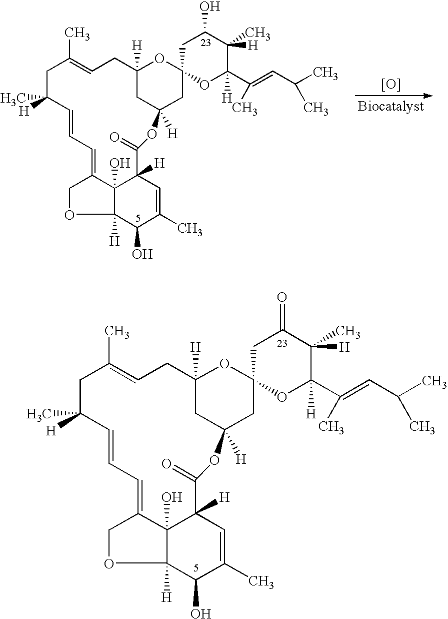

Lyophilized cells of Rhodococcus ruber DSM 44541 (0.6 g) are re-hydrated in phosphate buffer (6 mL, 50 mM, pH 8) for 30 minutes at 30° C. Co-substrate acetone (1 mL+2 mL after 6 h) and substrate LL-F8249-α (4.71 g, 7.7 mmol) are added and the mixture is shaken at 30° C. for 16 h. The reaction is stopped by centrifugation and extraction with ethyl acetate. The extracts are combined and concentrated under reduced pressure to afford the title product, which may used directly or purified by silica gel chromatography.

### EXAMPLE 5

### Preparation of Moxidectin

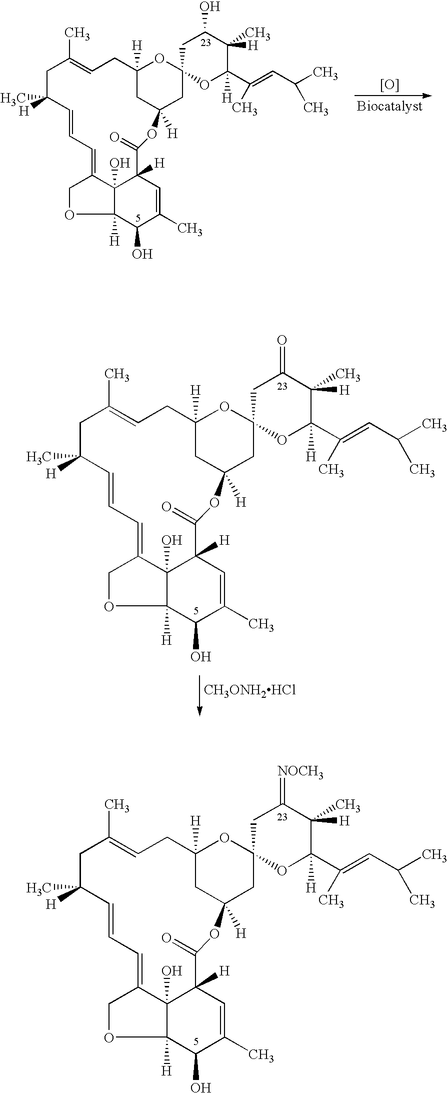

Lyophilized cells of Rhodococcus ruber DSM 44541 (0.6 g) are re-hydrated in phosphate buffer (6 mL, 50 mM, pH 8) for 30 minutes at 30° C. Co-substrate acetone (1 mL+2 mL after 6 h) and substrate LL-F8249-α (4.71 g, 7.7 mmol) are added and the mixture is shaken at 30° C. for 16 h. The reaction is stopped by centrifugation and extraction with ethyl acetate. The extracts are combined and concentrated to give a residue. The residue (2.44 g, 4.00 mmol of 23-(keto)-LL-F28249-α) is dissolved in dichloromethane, treated with a solution of methoxylamine hydrochloride (0.50 g, 6.00 mmol) and sodium acetate (0.49 g, 6.00 mmol) in water and stirred at 20°-25° C. for 10 hours. The phases are separated. The dichloromethane phase is washed with water dried over magnesium sulfate to give the title product as a solution in dichloromethane.

What is claimed is:

1. A process for preparation of 23-keto-LL-F28249-α which comprises reacting LL-F28249-α with a biocatalyst that is capable of specifically oxidising the 23-hydroxy group of LL-F28249-α.

2. The process according to claim 1 wherein said biocatalyst is a microorganism.

3. The process according to claim 1 wherein said biocatalyst is selected from the group consisting essentially of: a living microorganism; the spores of said microorganism; a dead microorganism; an extract of the cell contents of said microorganism; and an enzyme that converts LL-F28249-α into 23-keto-LL-F28249-α.

4. The process according to claim 2 wherein said microorganism is Rhodococcus ruber DSM 44541.

5. The process according to claim 2 wherein microorganism is a Streptomyces strain capable of specifically oxidising the 23-hydroxy group of LL-F28249-α.

6. The process according to claim 3 wherein said living microorganism is a whole cell microorganism in the form of vegetative cells, resting cells or freeze-dried cells.

7. The process according to claim 3 wherein said dead microorganism is in a partially disintegrated form with the cell wall/cell membrane mechanically or chemically permeabilized.

8. The process according to claim 3 wherein said enzyme is cloned and expressed or wild-type.

9. The process according to claim 8 wherein said enzyme is purified, partially purified or an extract.

10. A process for the manufacture of moxidectin which comprises reacting LL-F28249-α with a biocatalyst that is capable of specifically oxidising the 23-hydroxy group of LL-F28249-α to give the 23-keto-LL-F28249-α compound; and reacting said compound with methoxylamine or a salt thereof

11. The process according to claim 10 wherein said biocatalyst is a microorganism.

12. The process according to claim 10 wherein said biocatalyst is selected from the group consisting essentially of: a living microorganism; the spores of said microorganism; a dead microorganism; an extract of the cell contents of said microorganism; and an enzyme that converts LL-F28249-α into 23-keto-LL-F28249-α.

13. The process according to claim 11 wherein said microorganism is Rhodococcus ruber DSM 44541.

14. The process according to claim 11 wherein microorganism is a Streptomyces strain capable of specifically oxidising the 23-hydroxy group of LL-F28249-α.

15. The process according to claim 12 wherein said living microorganism is a whole cell microorganism in the form of vegetative cells, resting cells or freeze-dried cells.

16. The process according to claim 12 wherein said dead microorganism is in a partially disintegrated form with the cell wall/cell membrane mechanically or chemically permeabilized.

17. The process according to claim 12 wherein said enzyme is cloned and expressed or wild-type.

18. The process according to claim 17 wherein said enzyme is purified, partially purified or an extract.

19. The process according to claim 10 wherein said salt is a hydrochloride salt.
